(19) United States
(12) Patent Application Publication
(10) Pub. No.: US 2024/0296553 A1
(43) Pub. Date: Sep. 5, 2024

(54) Automatic Image Quantification from Physician-Generated Reports

(71) Applicant: Wisconsin Alumni Research Foundation, Madison, WI (US)

(72) Inventors: Tyler Bradshaw, Madison, WI (US); Zachary Huemann, Johnsburg, IL (US); Junjie Hu, Madison, WI (US)

(21) Appl. No.: 18/177,315

(22) Filed: Mar. 2, 2023

(51) Int. Cl. G06T 7/00; G06T 5/50; G06T 7/11; G06V 10/74; G16H 30/40; G16H 50/20

(52) U.S. Cl. CPC G06T 7/0012; G06T 5/50; G06T 7/11; G06V 10/761; G16H 30/40; G16H 50/20; G06T 2207/20221; G06T 2207/30061

(57) ABSTRACT

Physician-authored text descriptions of diagnostic images are used to inform an automatic segmentation and measurement of image features of that diagnostic image. The segmentations may be processed to extract quantitative values to then update the physician-authored text descriptions.

### STATEMENT REGARDING FEDERALLY SPONSORED RESEARCH OR DEVELOPMENT

--

### CROSS REFERENCE TO RELATED APPLICATION

--

### BACKGROUND OF THE INVENTION

The present invention relates to medical imaging and in particular to a system for detecting, segmenting, and quantifying measures of diagnostic images informed by a contemporaneous physician-authored report.

Medical images such as those obtained using x-ray, CT, MRI, or PET machines may be archived, for example, in a picture archiving and communication system (PACS) together with physician-generated reports. The report serves as an official record of the reading physician's interpretation of the medical image and is used to communicate findings to the patient and the patient's care team. The reports normally include a diagnosis and supporting quantitative data.

These reports may also be used to provide context for the interpretation of subsequent images. In these cases, the radiologist reviews the patient's prior images and prior reports to ascertain the location and extent of the disease and to assess its progress.

Recently, automated tools have been developed for analyzing medical images, for example, using machine learning systems. The internal operation of such machine learning systems is largely invisible, and although they can be statistically accurate, the possibility of profound, undetected errors make it difficult to get physicians and patients to trust such systems.

### SUMMARY OF THE INVENTION

The present invention leverages the existing physician-generated report to guide the automatic analysis of medical images, both allowing a broader range automatic analysis and reducing the opportunity for error in that analysis. In an important embodiment, the physician-generated report guides a segmentation that can be quickly reviewed by the physician prior to quantification, providing physician oversight to the automatic analysis while freeing the physician from the details of extracting quantitative values from the segmentation. The extensive experience of a human physician captured in the physician-generated report, together with the computational power that machine learning produces, can provide an analysis that is both accurate and trustworthy.

In one embodiment, the invention provides a system for automatic quantification of diagnostic images having a first input for receiving a current digitized medical diagnostic image of a patient and a second input for receiving a physician-authored text description based on the current digitized medical image and including references to a current quantitative measure of the current digitized medical diagnostic image. A machine learning system receives the current digitized medical diagnostic image and the physician-authored text description to determine the current quantitative measure. A text editor then links the references to a quantitative measure in the previously prepared physician-authored text descriptions to the determined current quantitative measure.

It is thus a feature of at least one embodiment of the invention to guide a machine learning system in analyzing a medical image by a draft physician report.

The machine learning system may have weights trained with a training set of physician-authored text descriptions and image segmentations for images providing the basis of the physician-authored text descriptions and generates a segmentation of the current digitized medical diagnostic image.

It is thus a feature of at least one embodiment of the invention to provide a machine learning system trained to combine text and images to produce image segmentations.

The system may include a geometric calculator receiving the generated segmentation and providing the determined current quantitative measure based on the segmentation.

It is us a feature of at least one embodiment of the invention to provide a system that can augment the physician-generated reports that are usually linked to medical images in archiving systems.

The system may further provide an output outputting a file for storage linking the current digitized medical diagnostic image to the physician-authored text description based on the current digitized medical image, the determined quantitative measure, and the segmentation file storage system.

It is thus a feature of at least one embodiment of the invention to augment an archived medical image with a segmentation image.

The text editor may insert the determined current quantitative measure into the physician-authored text description of the current digitized medical diagnostic image.

It is thus a feature of at least one embodiment of the invention to integrate data extracted by a machine learning system into the physician report for convenient review and storage.

The system may include an image display displaying the current digitized medical diagnostic image superimposed with a representation of the determined current quantitative measures for physician confirmation.

It is thus a feature of at least one embodiment of the invention to permit physician review of the automatic analysis in the context of the image.

The segmentation of the current digitized medical diagnostic image may be superimposed on the current digitized medical diagnostic image.

It is thus a feature of at least one embodiment of the invention to expose at least one intermediate step in the automatic analysis of the image that can be confirmed quickly by visual inspection by the physician.

In some embodiments, the system may provide a third input for receiving a prior quantitative measure related to a prior digitized medical image of a same patient as the current digitized medical image. A comparator may output a trend value indicating changes between the determined quantitative measures (of the current digitized medical image) and the prior quantitative measures linked by the text editor to the current quantitative measure.

It is thus a feature of at least one embodiment of the invention to permit automatic trend extraction.

The third input may be provided by the machine learning system receiving the prior digitized medical image and a prior physician-authored text description based on the prior digitized medical image.

It is thus a feature of at least one embodiment of the invention to provide trend analysis even when quantitative values are not associated with prior images.

The machine learning system may use an encoder-decoder neural network

It is thus a feature of at least one embodiment of the invention to augment a robust segmentation architecture with language model data.

These particular objects and advantages may apply to only some embodiments falling within the claims and thus do not define the scope of the invention.

### BRIEF DESCRIPTION OF THE DRAWINGS

FIG. 1 is a block diagram of the principal components of the present invention including a trained machine learning system receiving diagnostic images and physician-generated reports providing descriptions of those images;

FIG. 2 is a logical representation of a file linking the physician text descriptions with inserted quantitative values together with a diagnostic image and a segmented diagnostic image used for the quantitative values;

FIG. 3 is a fragmentary detail of an alternative embodiment of FIG. 1 providing trend information by review of historical physician-generated reports and diagnostic images;

FIG. 4 is a fragment of a physician-generated report augmented with trend data using the technique of FIG. 3; and

FIG. 5 is a block diagram of a machine learning system suitable for use with the present invention.

### DETAILED DESCRIPTION OF THE PREFERRED EMBODIMENT

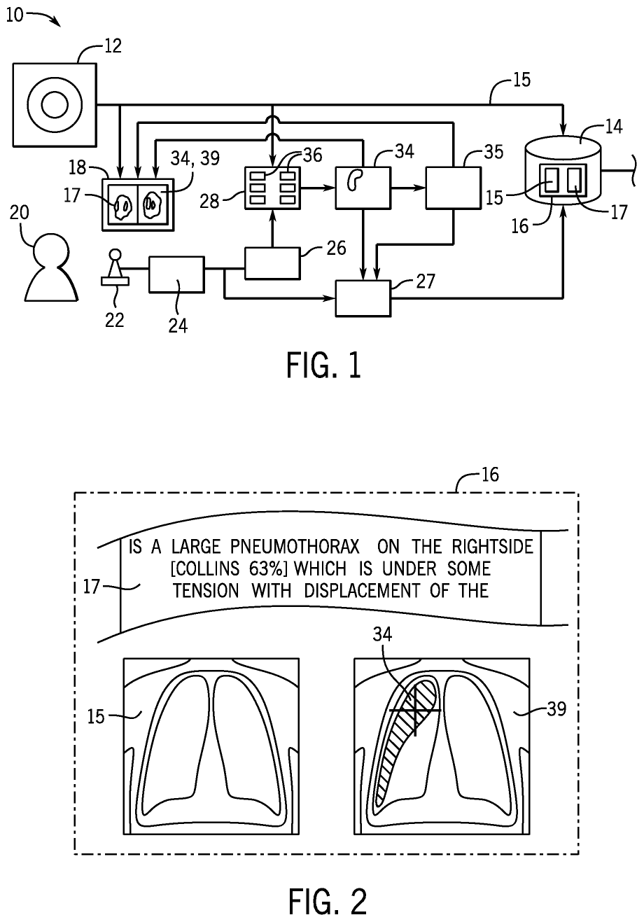

Referring now to FIG. 1, an automatic diagnostic image quantification system 10 may make use of data from one or more diagnostic imaging machines 12, for example, x-ray machines, magnetic resonance imaging machines (MRI), computed tomography machines (CT), or positron emission tomography machines (PET). These machines 12 provide digitized medical diagnostic images 15 of a patient that may be communicated, for example, to a database system 14 holding multiple files 16 linking the data of the digitized medical diagnostic images 15 with physician-generated reports 17. The diagnostic images 15 may be two-dimensional arrays of pixel values or three-dimensional arrays of voxel values as is generally understood in the art.

A given digitized medical diagnostic image 15 may be displayed on a terminal 18 providing a graphic display of the diagnostic image 15 allowing review by a physician 20. Based on this review the physician 20 may prepare a preliminary physician report 26 describing clinically relevant features of the diagnostic image 15. In one nonlimiting embodiment, the physician 20 may provide dictation into a microphone 22 for conversion into text by a speech-to-text engine 24 to produce this preliminary physician report 26.

The preliminary physician report 26 may refer to various structures or features of the diagnostic images 15 without providing a quantitative value for those structures or features. For example, a preliminary physician report 26 may include the text fragment of:
There is a large pneumothorax on the right side [ ] which is under some tension, with displacement of the mediastinum toward the opposite side.

Optionally, this text fragment may include tags (e.g., “[ ]”) entered by the physician 20 indicating locations where a quantitative value is desired. Alternatively, these quantitative values and their location may be inferred in the processing to be described below.

Referring still to FIG. 1, the preliminary physician report 26 and the current digitized medical diagnostic image 15, being reviewed by the physician 20 during the dictation of that preliminary physician report 26, may be provided to a machine learning system 28 to be processed according to model weights 30 developed by training as will be discussed below.

Using these inputs, the machine learning system 28 outputs a segmentation image 34 of the current digitized medical diagnostic image 15, for example, quantitatively describing a volume of an organ, a lesion, or other body structure mentioned in the preliminary physician report 26. The segmentation image 34 may be provided to the terminal 18 for review by the physician 20 to ensure that this automatic process correctly identified the desired anatomy as can be determined quickly by visual inspection. If approved by the physician 20 (for example, through a keyboard or other device associated with the terminal 18), the segmentation image 34 is provided to a geometric calculator 35 which performs basic calculations to distill the segmentation image 34 into a single or a few quantitative values.

In one example, and referring to FIG. 2, the segmentation image 34 may identify a pneumothorax or collapsed lung whose volume deficiency may be highlighted and registered with the image 15 to produce a composite image 39, supplementing or used in place of the segmentation image 34 to assist visual confirmation by the physician 20. This composite image 39 may optionally be stored in the file 16 in lieu of or together with the segmentation image 34.

When the segmentation image 34 is a pneumothorax, the geometric calculator 35 may, for example, employed the Collins method to characterize the pneumothorax in a single percentage value. The results of this calculation may then be inserted by a text editor 27 into the preliminary physician report 26, for example, characterizing the pneumothorax as [Collins 63%] as depicted in FIG. 2 to produce the physician-generated report 17 fragment:
There is a large pneumothorax on the right side [Collins 63%] which is under some tension, with displacement of the mediastinum toward the opposite side.

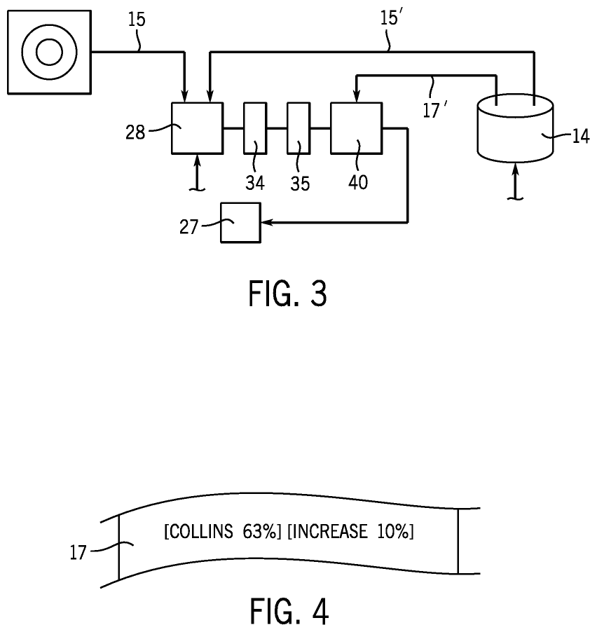

Referring now to FIG. 3, in an alternative embodiment, machine learning system 28, in addition to receiving a current diagnostic image 15, may also receive a previously acquired diagnostic image 15′ and associated physician-generated reports 17′ to determine a general trend in the progress of a medical condition. The current quantification of the segmentation associated with the current diagnostic image 15 may be inserted into the text file (e.g., [Collins 63%]) and this value also compared to a corresponding value deduced from a previously acquired diagnostic image 15′ (prepared using the same techniques of analysis described above) or obtained directly from the previous physician-generated report 17′. This comparison process may be performed by a comparator 40 provides a trend value (e.g., [increased 10%]) that may also be inserted into the physician-generated report 17 as shown in FIG. 4 and simply linked by text editor 27 to the quantification derived from the current diagnostic image 15 and current physician-generated report 17.

Analysis of the previous diagnostic image 15′, using the techniques described above, may also produce a segmentation of the previous diagnostic image 15′ which may be registered with the current diagnostic image 15 to guide the physician.

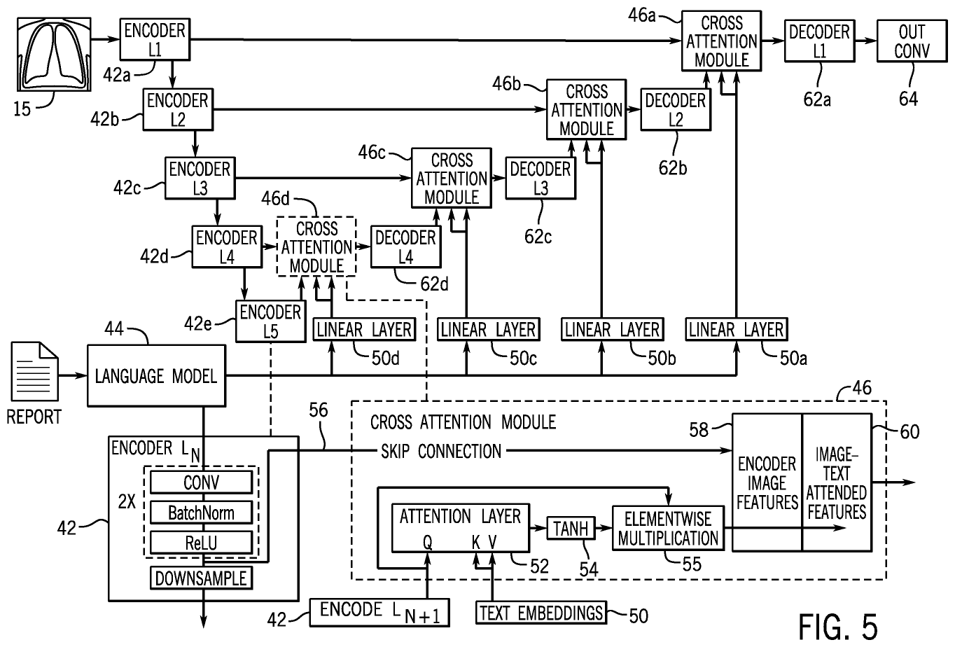

Referring now to FIG. 5 in one embodiment, the machine learning system 28 may employ the framework of an encoder-decoder network such as a U-NET, for example, described in Ronneberger, O., Fischer, P., Brox, T. (2015), U-Net: Convolutional Networks for Biomedical Image Segmentation, In: Navab, N., Hornegger, J., Wells, W., Frangi, A. (eds) Medical Image Computing and Computer-Assisted Intervention-MICCAI 2015. MICCAI 2015, Lecture Notes in Computer Science( ), vol 9351. Springer, Cham. https://doi.org/10.1007/978-3-319-24574-4_28, hereby incorporated by reference.

Using this framework, segmentation features are extracted from the diagnostic image 15 via a set of encoders 42a-42e providing successive compressions to the diagnostic image 15 based on weights 30 derived during training. Each encoder 42 provides two sets of convolutions with batch normalization and rectified linear unit (ReLU) activation. The output of each encoder 42 is fed to a corresponding cross-attention module 46a-46d and then downsampled via max pooling and fed to the next lower encoder 42.

To integrate language into the model, first, a textual vector representation is extracted from the text via a language model 44. An example language model is T5-Large described at Raffel, C., Shazeer, N., Roberts, A., Lee, K., Narang, S., Matena, M., Zhou, Y., Li, W. & Liu, P., Exploring the Limits of Transfer Learning with a Unified Text (arXiv, 2020 Jul. 28), http://arxiv.org/abs/1910.1068, hereby incorporated by reference. This textual vector representation may then be reduced in dimensionality by a projection head 50a-50d to match the dimensionality of the encoding level. The T5-large language model 44 may, in one example, be pre-trained on the c4 dataset which contains roughly 300 G of text, and a series of tasks, text classification, question answering, machine translation, and abstractive summarization.

The textual information from the language model 44 is injected into the processing of the diagnostic images 15 by means of a set of cross-attention modules 46, which operate on the upsampled decode feature maps. The language cross-attention of the cross-attenuation modules 46 uses a single-headed attention module 52 in which the output from a given encoder 42 for a given level is the query and the textual vector, as processed by the projection heads 50, is the key and value pair. This output produces a pixel-wise attention map.

The pixel-wise attention map, A, is obtained by:

$$A = softmax ( \frac{Q {{W}^{Q} ( K {W}^{K} )}^{T}}{\sqrt{{d}_{k}}} ) V {W}^{V} \qquad ( 1 )$$

where A is the pixel-wise attention map, Q is the upsampled feature map, K and V are the projected language vectors, and W^{Q}, W^{K}, and W^{Y} are the learned attention weights.

These vectors need to be reshaped to be used in Eq.1, specifically flattened into:

Q∈ [symbol] ^{w×h×c}→Q∈ [symbol] ^{wh×c} (2)
where w is the width, h is the height, and c is the number of channels in the layer.

Additionally K and V both share the same dimensions as they are the same vector repeated, specifically:

K,V∈ [symbol] ^{seq}^{ᵢ}^{×emb}^{ₚ} (3)
where seqᵢ is the sequence length of 512 for all language models used and embₚ is the projected embedding length which is chosen to match the number of channels c at each layer of the U-Net.

The output of the attention module 52 is compressed using a tanh function 54 to pixel values between −1 and 1. This is then used to obtain the attention weighted decode feature map 60 by doing pixel-wise multiplication 55 of the of the normalized attention weights and the decode feature map 42 calculated as:

$$\hat{Q} = tanh ( A ) * Q \qquad ( 4 )$$

where * is the element-wise multiplication of the input feature map and the normalized pixel attention map, and \hat{Q} is the attention-weighted feature map.

The diagnostic image quantification system 10 may be trained with the teaching set comprised of physician-generated reports and associated diagnostic images and segmentations of those images. During this training the weights of the language model 44 are frozen and the physician-generate reports 15 of the training set are applied to the language model 44 and the hidden state vectors of the language model 44 used as output. The output representation has dimensions of 512×1024 (token length×embedding dimension) for T5-Large. This output then goes through the corresponding projection heads 50 to lower the embedding dimensionality of the hidden state vector to match the number of channels in the encoder feature maps. This step allows the language cross-attention to work at multiple levels in the U-Net decoder. During training, the weights of the language model 44 are frozen. This prevents the loss incurred from segmentation errors from propagating to the language model which would often destroy its ability to extract useful language features.

In one embodiment, segmentation for pneumothorax may use the CANDID-PTX dataset consisting of 19,237 chest radiographs with reports and segmentations of pneumothoraces, acute rib fractures, and intercostal chest tubes.

The various components described above may be implemented on appropriate hardware, for example, as used for machine calculations and machine learning as is understood in the art.

Certain terminology is used herein for purposes of reference only, and thus is not intended to be limiting, for example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112(f) unless the words “means for” or “step for” are explicitly used in the particular claim.

What we claim is:

1. A system for automatic quantification of diagnostic images comprising:
a first input for receiving a current digitized medical diagnostic image of a patient;
a second input for receiving a physician-authored text description based on the current digitized medical image and including references to a current quantitative measure of the current digitized medical diagnostic image;
a machine learning system receiving the current digitized medical diagnostic image and the physician-authored text description to determine the current quantitative measure; and
a text editor receiving the physician-authored text descriptions and linking the references to a quantitative measure to the determined current quantitative measure.

2. The system of claim 1 wherein the machine learning system provides weights trained with a training set of physician-authored text descriptions and image segmentations for images providing a basis of the physician-authored text descriptions and generates a segmentation of the current digitized medical diagnostic image.

3. The system of claim 2 further including a geometric calculator receiving the generated segmentation and providing the determined current quantitative measure based on the segmentation.

4. The system of claim 2 further including an output outputting a file for storage linking the current digitized medical diagnostic image to the physician-authored text description based on the current digitized medical image, the determined quantitative measure, and the generated segmentation.

5. The system of claim 1 wherein the text editor inserts the determined current quantitative measure into the physician-authored text description of the current digitized medical diagnostic image.

6. The system of claim 1 further including an image display displaying the current digitized medical diagnostic image superimposed with a representation of the determined current quantitative measures for physician confirmation.

7. The system of claim 1 wherein the machine learning system provides weights trained with a training set of physician-authored text descriptions and image segmentations for images providing a basis of the physician-authored text descriptions and generates a segmentation of the current digitized medical diagnostic image, and
wherein the image display further displays generated segmentation superimposed on the current digitized medical diagnostic image.

8. The system of claim 1 further including a third input for receiving a prior quantitative measure related to a prior digitized medical image of a same patient as the current digitized medical image; and
further including a comparator outputting a trend output indicating changes between the determined quantitative measures and the prior quantitative measures' output values from the prior output values; and
wherein the text editor links the trend output to the current quantitative measure.

9. The system of claim 8 wherein the third input is provided by the machine learning system receiving the prior digitized medical image and a prior physician-authored text description based on the prior digitized medical image.

10. The system of claim 1 further including a third input for receiving a prior digitized medical image of a same patient as the current digitized medical image; and wherein the machine learning system provides weights trained with a training set of physician-authored text descriptions and image segmentations for images providing a basis of the physician-authored text descriptions and generates a segmentation of the prior digitized medical diagnostic image;
further including an image display displaying the generated segmentation superimposed on the current digitized medical diagnostic image.

11. A method of automatic quantification of diagnostic images comprising:
(a) receiving a current digitized medical diagnostic image of a patient;
(b) receiving a physician-authored text description based on the current digitized medical image and including references to a current quantitative measure of the current digitized medical diagnostic image;
(c) using a machine learning system receiving the current digitized medical diagnostic image and the physician-authored text description to determine the current quantitative measure; and
(d) receiving the physician-authored text descriptions and linking the references to quantitative measures to the determined current quantitative measure.

12. The method of claim 11 wherein the machine learning system provides weights trained with a training set of physician-authored text descriptions and image segmentations for images providing a basis of the physician-authored text descriptions and generates a segmentation of the current digitized medical diagnostic image.

13. The method of claim 12 further including receiving the generated segmentation and providing the determined current quantitative measure based on the segmentation.

14. The method of claim 12 further including outputting a file for storage linking the current digitized medical diagnostic image to the physician-authored text description based on the current digitized medical image, the determined quantitative measure, and the generated segmentation.

15. The method of claim 11 including inserting the determined current quantitative measure into the physician-authored text description of the current digitized medical diagnostic image.

16. The method of claim 11 further including displaying the current digitized medical diagnostic image superimposed with a representation of the determined current quantitative measures for physician confirmation.

17. The method of claim 15 wherein the machine learning system provides weights trained with a training set of physician-authored text descriptions and image segmentations for images providing a basis of the physician-authored text descriptions and generates a segmentation of the current digitized medical diagnostic image, and
and including displaying the generated segmentation.

18. The method of claim 11 further including:
receiving a prior quantitative measure related to a prior digitized medical image of a same patient as the current digitized medical image;
outputting a trend output indicating changes between the determined quantitative measures and the prior quantitative measures' output values from the prior output values; and
linking the trend output to the current quantitative measure.

19. The method of claim 18 including using the machine learning system to determine the prior quantitative measure from the prior digitized medical image and a prior physician-authored text description based on the prior digitized medical image.

20. The method of claim 11 further including receiving a prior digitized medical image of a same patient as the current digitized medical image to generate a segmentation of the prior digitized medical diagnostic image; and
displaying the generated segmentation superimposed on the current digitized medical diagnostic image.
